(19) United States
(12) Patent Application Publication
(10) Pub. No.: US 2023/0235196 A1
(43) Pub. Date: Jul. 27, 2023

(54) ANTI-BACTERIAL AND/OR ANTI-VIRAL TAPE

(71) Applicants: Kegham Alex DANTZIGUIAN, Dubai (AE); A. Hagop KASPARIAN, Brussels (BE)

(72) Inventors: Kegham Alex DANTZIGUIAN, Dubai (AE); A. Hagop KASPARIAN, Brussels (BE)

(21) Appl. No.: 18/008,951

(22) PCT Filed: Jun. 9, 2021
(86) PCT No.: PCT/EP2021/065457

§ 371 Date: Dec. 7, 2022

(30) Foreign Application Priority Data
Jun. 9, 2020 (EP) 20178928.6

(51) Int. Cl. C09J 7/20; A61K 33/30; A61K 33/34; A61K 33/38; A61P 17/02

(52) U.S. Cl. CPC C09J 7/201; A61K 33/30; A61K 33/34; A61K 33/38; A61P 17/02; C09J 2301/16; C09J 2301/41; C09J 2301/302

(57) ABSTRACT

The invention relates to a multilayer flexible material (of a tape type) which can be applied and adhere onto a surface to modify its properties. Particularly, an antibacterial and/or antiviral multilayers material comprising a tie binding layer with at least one adhesive face arranged to be fixed on any suitable surface and an active treatment layer arranged on the face of the tie binding layer opposite to adhesive face, said treatment layer comprising anti-bacterial and/or anti-viral agents. The invention is also related to an associated application method and specific uses.

### FIELD OF THE INVENTION

The invention relates to a multilayer flexible material, of a tape type, which can be applied and adhere onto a surface to modify its properties. In particular, a modifying combination of materials in solid and/or different states is deposited onto the surface which can for example make the surface auto sanitizing, perpetually or continuously active, with a broad range and quick acting anti-bacterial and/or anti-viral activity.

The anti-viral activity advantageously comprises for example anti Corona and/or other anti-virus activity, or used as an antiseptic dressing to protect from antibiotic resistant organisms.

### BACKGROUND INFORMATION

The existing antibacterial antiviral products to treat surfaces that are prone to be touched by many people mainly are paints or coatings. The film these types of paints form are blend combinations of active biocides binders and fillers.

Due to nature of paints, the biocides use antibacterial antiviral ingredients which are covered by the binder and/or other paint components, that reduce the active ingredients performance with time.

In addition, as in most cases they are in liquid form, the antibacterial, antiviral ingredients are difficult to put into suspension and to apply, hence the limitation of binders that can be used.

The other components in the paint limit the elimination of a broad range of antibacterial and antiviral materials. Making them inflexible and not safe enough. In particular, the following problems are encountered:
I. Weathering limitation.
II. Limitation of active antibacterial antiviral thickness, exposure surface area.
III. The active part are not monolithic. The surface obtained is a blend of various materials.
IV. Since most paints are organic, they themselves are breeding ground for organic growth.
V. Nature and method of application dictates that to produce the paint; various components are usually ground down to make it possible to be used as a paint, make it compatible and be in suspense and be possible to apply.
VI. In most cases the existing film thickness is limited and therefore can be applied only on limited selected type of surfaces.
VII. The vehicles used in most cases have an odor, most release volatile material and can be irritant people can develop an allergy or illness from it.
VIII. Limitations in adjusting with anti bacterial and/or anti viral requirements, as ingredients that can act as anti-bacterial and/or anti-viral may not be compatible with other ingredients. Difficulty in formulation
IX. Limitations in performance, technical rigidity to adjust to different emerging pathogens such as other neutralizing bacteria virus systems, technically difficult to be bioelectric, moisture absorbing, etc.
X. Application of paints are time consuming, tedious, require time to cure, hence treated units cannot be used for some time. In addition, they require certain skills and not to effect surroundings. Are messy to use.
XI. Limited by time, difficult to remove.
XII. Difficult to apply uniformly.
XIII. Requires application tools.
XIV. Limited in exposure durability in harsh climatic conditions.

The applicant therefore made an anti-bacterial/viral tape that eliminates at least part of the above shortcomings and offers solutions in all the above cases, such as ease of application, instant usage, require practically no skill, ability to evolve to meet newer emerging pathogens, non allergenic.

### INVENTION DETAILS

The invention concerns an antibacterial and/or antiviral multilayers material comprising:
a tie binding material layer with at least one adhesive face arranged to be fixed on any suitable surface, and
an active treatment layer comprising anti-bacterial and/or anti-viral agents.

In one aspect of the invention, the active treatment layer is arranged on the face of the tie binding layer opposite to adhesive face. This enables to have an external surface with autosanitizing properties.

In another aspect of the invention, the active treatment layer is arranged to cover at least in part the adhesive face of the tie binding layer. This arrangement enables to decontaminate the substrate, the material being then removed after treatment. This arrangement is also suitable for wound dressings, where the antibacterial has to be on the skin side (the skin being here the substrate).

In this application, “surface to be treated” is also referred to as “substrate”.

The material of the invention is an easy-to-use material that can be applied onto surfaces to make the surface continuously self-sanitizing, as having perpetual antibacterial and/or antiviral properties.

The objective has been to create ‘SMS’ (Selected Material Surfaces), to change various surfaces (Various physical states) to have designed/engineered purposes, such as changing sub surfaces to other desired physical, chemical, biological properties. In addition to have the newly obtained surface to perform as per desired and designed performance.

To easily be shaped to that of the substrate.

To achieve the above, various “tie binder” materials have been created, to tie to the surface to be treated the desired designed ‘Surface (1)’, i.e., the desired properties, with consideration that the ‘Tie binder’ does not affect/deteriorate in any way the ‘Selected material surface (1)’.

The ‘Tie binder's’ purpose is to join the subsurface (surface to be treated) to the newly obtained ‘Surface (1)’.

The tie binding layer is a thin layer material having two sides, one side is used to adhere to the active material (the exposed part), the other side is used to bind to the desired substrate (surface to be treated). The tie binding layer can be an assembly of several layers, for example a structural layer and an adhesive layer.

In certain applications the active material is made to be in contact with the substrate. In such applications, the tie binding layer is made to have single adhering surface, where part of this surface adheres to the active material and the rest of the surface adheres to the substrate, hence the active material can be made to be in between the single side binding layer and substrate, also if desired various in between material are used, this application is used to disinfect the substrate and if required can easily be removed. The active material as used in this invention performs against antibiotic resistant pathogens, in addition being non allergic, nonirritating, continuously active, the inner surface of the active material continuously is biologically clean.

The tie binding layer is therefore a structural layer intended to link the active layer and the substrate.

The Tie binders preferably is or comprises a pressure sensitive adhesive for example such as: PET, PVC, fabric with a rubber adhesive system backing, foamed pure acrylic, foamed modified acrylic, solid pure acrylic or others such as polyurethane, polyurea, epoxy, cyanoacrylate, PVA, contact adhesive, hot melt materials or a combination thereof.

The newly obtained surface (the external surface of the tape with antibacterial and/or antiviral properties) will have its own desired properties and performance.

The surface to be treated onto which the material of the invention has been applied is the “newly obtained surface”.

Further, this newly obtained surface can be used as a subsurface that can be used to enhance, interact with an additional added second surface using the same tie binder or another purposely made second tie binder, enabling both surfaces to act in synergy. If needed, further similar several built-up other designed surfaces can be added. The surfaces can for example be also interwoven, and not necessarily one on top of the other, or can also be along side by side in a pattern (Repetition of built up can be done if necessary). In other words, the multilayers adhesive material of the invention allows the combination of several tapes (with different compositions if required) with specific arrangement (one on top of the other or side by side for instance).

In some instances, as mentioned earlier, the active material is made to be in direct contact with the subsurface, where it acts as a disinfectant, for example as wound dressings. Thanks to medical research centers worldwide, materials that act as anti-bacterial and anti-viral are well identified and known, in addition various other well-known process(s) for further enhancements are well documented, the invention transforms these materials, process(s) to a practical product to treat various surfaces, to make them auto sanitizing, continuously antibacterial and antiviral.

The composition of the active layer is a proven biocide against current known bacteria and/or viruses. The composition can comprise a metallic agent, i.e., a metal or metal alloy that performs by oligodynamic effect, like for example copper and/or copper alloys, silver, brass, bronze, zinc, zinc oxide, titanium dioxide and/or a combination thereof.

The active layer can be in foil, mesh, flakes, powder or a composition of several forms, plus in some cases with or without additional combination of components such as silver ion zeolites, zinc ion zeolites, copper ion zeolites, nano titanium dioxide, moisture absorbing and/or other biocides.

The active layer may—also comprise physical or aesthetic enhancers, patterns, or embossments. The composition is selected in function of the type of expected contamination to be neutralized.

The active layer can be deposited directly on the tie binding layer and glued onto. Indeed, the layers are fixed together by the pressure adhesive properties of the tie binding layer.

In certain cases, where the active material is fragile (taking into account manufacturing and handling), this active material can be deposited initially on a sacrificial film to have better manufacturing mechanical properties, which can be disposed off after the actual material is created.

However, in certain applications (used as a dressing isolating), absorbing material such as ‘Gauze’ can be used as an intermediate layer.

If the active layer is a combination of agents, the materials may be perforated in such a way that compounds can be in contact with the tie binding layer.

The created product(s) and the system is flexible enough to adapt to various different conditions and needs, in addition it allows the desired surface to be made from multi component functions in combination of various systems that work together in synergy to neutralize a wide range of bacteria and viruses.

The created product can be designed for bioelectric needs, have photo catalytic properties, contain moisture absorbing ingredients, physical neutralizing materials, the antibacterial antiviral material(s)can be the basis of the active layer.

In addition, it is also known that certain types of material can better perform in sequence as antibacterial and antiviral, in these types of requirements the result is built up in combination of surfaces that act in synergy.

The obtained surface can of be, powder, granules, foils, flakes, shapes, size and pattern, some inlaid, embossed for improving performance in addition increasing the exposed surface areas and/or for esthetic purposes.

### Application Method

Methods and conditions of application depend on the substrate to be treated and the type of ‘Tie binder’ needed.

In most cases surfaces to be treated are architectural components, like handles or door, the need is the simplicity and ease of application.

The invention will be better understood in relation with the figures where:

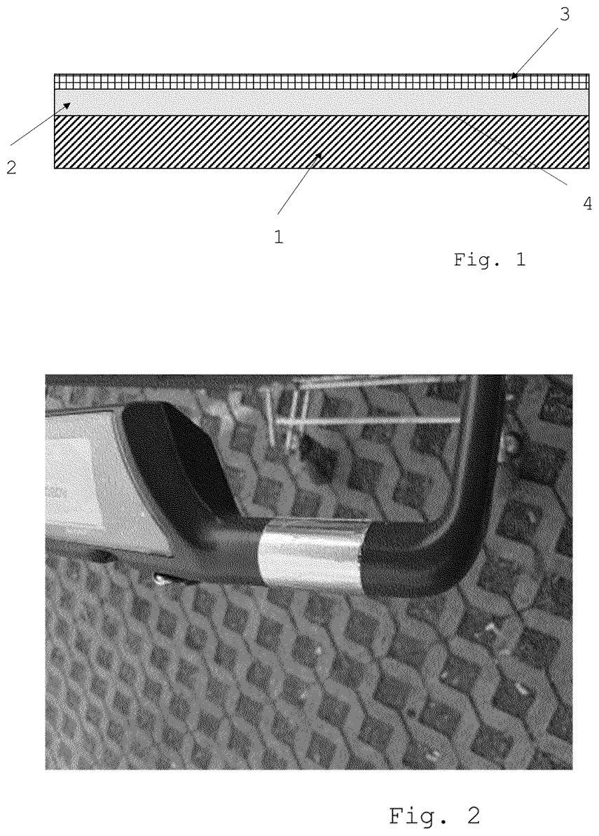

FIG. 1 is a schematic view of the arrangement of a multilayer material according to the invention.

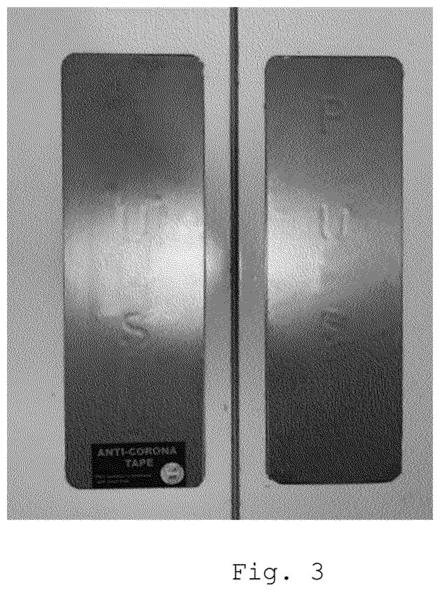

FIG. 2 is a picture of a trolley handle covered with a multilayer material according to the invention;

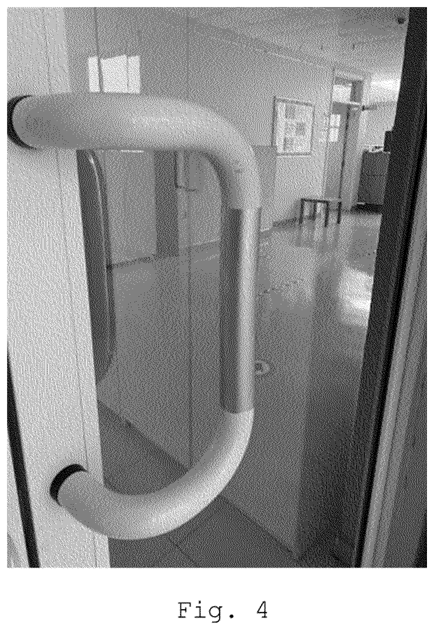

FIG. 3 is a picture of a door partially covered with a multilayer material according to the invention;

FIG. 4 is a picture of a door handle covered with a multilayer material according to the invention;

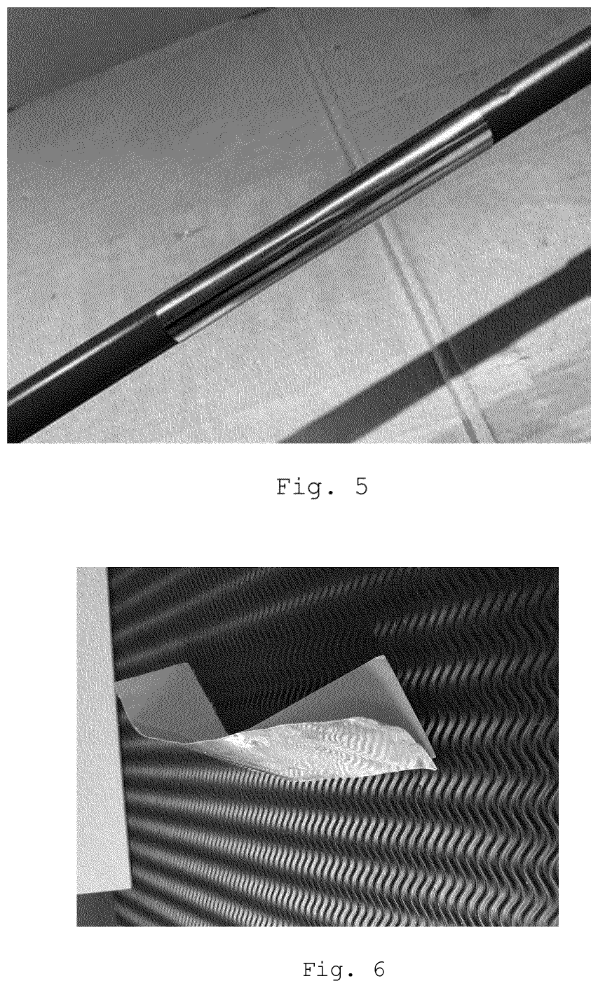

FIG. 5 is a picture of a broomstick partially covered with a multilayer material according to the invention.

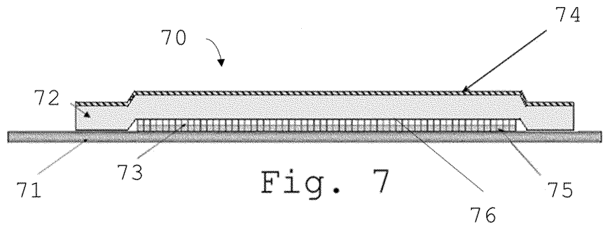

FIG. 6 is a picture of the tape where the disposable protective layer is partially removed.

FIG. 7 is a schematic view of the arrangement of a multilayer material according to the invention for sub surface protection.

According to FIG. 1, the multilayers adhesive material comprises a tie binding layer 2 taped to a substrate 1 thanks to adhesive surface 4. The active treatment layer 3, disposed directly on the layer 2, is external and covers entirely the substrate 1.

The multilayers adhesive material of the invention can be in form of a roll or in sheets of specific dimensions.

The ‘Tie binder’ 2 in this case is a type of pressure sensitive tape. To apply the multilayers adhesive material, the surface to be treated 1 is first cleaned from material that may affect the bonding of the ‘Tie binder’. Then the disposable protective film is removed (depending if in roll form or not) from the multilayers adhesive material that is spread over the surface to be treated, the substrate 2, for example by unrolling the roll of multilayers adhesive material, and then firmly and neatly pressed onto the substrate to ensure adhesion of the material to the substrate. The treatment layer is then the outer layer.

The multilayers adhesive material can further comprise a protective film, to protect the exposed adhesive side of the tie binding layer. In that case, the protective film is removed from the ‘Tie binder’ (2) and disposed of before spreading the material over the substrate. When the material of the invention is presented as a roll, a protective film may not be necessary.

The multilayers adhesive material can further comprise a protective film, to protect the exposed active layer from degradation or scratches during storage or transport. This film is advantageously removed after placement on the substrate.

The newly obtained ‘Surface (1)’ acts as an auto neutralizing and continuously anti-bacterial and anti-viral surface.

In other more demanding conditions, the ‘Tie binder’ can be a variety of bonding materials, that may be in liquid, solid, paste, or multi-component forms.

According to FIG. 7, the multilayers material 70 of the invention is made to protect as an antiseptic tape. The binding layer 72 has a adhesive surface 76 on which is applied an active treatment layer 73 having a smaller area than the adhesive surface 76. A neutral insulant layer (such as for example gauze or cotton) is applied onto the active treatment layer 73. Layer 74 is applied on the layer 72 opposite to the adhesive surface 76 to protect against elements or insure impermeability.

This multilayer 70 is applied on skin 71.

The ‘Tie binder’ 72 in this case is a type of pressure sensitive tape. To apply the multilayers adhesive material, the surface to be treated 71 (the skin) is first cleaned from material that may affect the bonding of the ‘Tie binder’. Then the multilayers adhesive material is placed over the surface to be treated, the substrate 71 and then firmly and neatly pressed onto the substrate to ensure adhesion of the material through surface 76 to the skin 71. The treatment layer 73, 75 is then against the skin.

The multilayers adhesive material 70 can further comprise a protective film (here not shown), to protect the exposed adhesive side of the tie binding layer before use. In that case, the protective film is removed from the ‘Tie binder’ 72 and disposed off before applying the material over the skin 71.

The multilayers adhesive material can further comprise a protective layer 74, to protect the exposed active layer from degradation or scratches during storage and protection layer against external elements.

The tape 70 here acts as an auto neutralizing and continuously anti-bacterial and anti-viral protection of the skin 71 in this case of application per FIG. 7 acts as a wound antiseptic dressing.

Tie binders are made to be used in such a way that they do not affect or reduce the performance of the desired and designed surface and act just as an inter-bond between two materials.

The ‘Tie binders’ are selected as per binding performance needs. Certain ‘Tie binders’ can be suitable with certain active ingredients and surfaces to be treated, and other ‘Tie binders’ are selected to be suitable to other active ingredients.

For more precision in application, the active ingredient(s) is applied onto the tie binding layer using methods such as: laser, electromagnetic, multi 3d, stamp, transfer, screen printing, CVD, or simple pressure.

In case a multi component is required, then these can be applied by various well-known application techniques and machines.

### Problems Solved by this Invention

Makes surfaces continuously self-sanitizing, antibacterial and/or antiviral for at least for a very long period of time or even perpetually.
In most cases it is practical, easy to use and apply, does not require special tools, simple to handle, that can be applied on a wide ranging of flat, curved, or odd shaped surfaces and materials, that is robust, durable, odorless, fast to apply and unit put into usage.
Requiring no solvent to apply.
Neutralizes ‘Dry’ contaminations.
The newly obtained surface is easy to maintain.
In addition, the technique offers solutions for more complex and demanding requirements, such as to neutralize bacteria and viruses in shorter periods of time.
It is also flexible enough to be modified to perform against bacterial or viral contaminates which may not yet be identified or discovered to date.

The key innovative features of the invention

The multilayer material of the invention can be available in several forms, depending on the method of application, substrate, and use. One of its forms is made to be simple to use, maintain, and apply on a variety of surfaces. It's made ready to use with a pressure sensitive tie binder which has a protective foil that is removed and disposed of prior to application. Then the tie binder area is pressed against the surface to be treated. Within 72 hours, the tie binder achieves its total bond strength. The newly obtained treated surface is quickly and continuously acting as a self-sanitizing antibacterial and/or antiviral. In addition, the newly obtained surface auto eliminates further bacteria and viruses with time.

The newly obtained surface requires no maintenance except routine dry or damp wiping with a soft cloth to clean the surface from soiling.

There are other variations of the basic unit, several combinations of the active antibacterial and/or antiviral components and tie binder(s) that are designed taking into consideration the application method and type of surface to be treated and required performance and exposure conditions.

### Method of Manufacturing the Invention

Using a liquid and/or solid pressure sensitive ‘Tie bond’, various ingredients were dropped, spread, and applied on the ‘Tie bond’ surface.

The tie bonds were selected depending on:
Effectiveness to bind active ingredients to various surfaces.
Durability.
Abrasion resistance.
Odorless.
Environmental conditions (Rain, Dry, Humid, Extreme cold, Extreme Heat).
Easy to use.
Safe for the applicator and user.

The selection of the neutralizing ingredients on the newly obtained surface are made:
Based on their neutralizing effectiveness on bacteria and/or viruses and as per the guidance and results of published information of medical and laboratory research centers.
The most difficult to neutralize contaminates.
Safety in day-to-day usage for the applicators and users.
Durability of surface in day-to-day usage.
Resistant to a variation of temperatures and other climatic conditions.
Esthetics.

In one embodiment of the invention, the multilayers adhesive material comprises an active layer being pure monolithic 100% Copper, made with various thicknesses. Preferably, the thickness of copper is between 0.02-0.08 mm. These values correspond to expected exposure and substrates shape. The binding layer used can be a tear resistant flexible fabric backing with a rubber adhesive system. Alternatively, the binding layer can be a PET backing and a tackifier acrylic adhesive or a PVC-film backing and a tackifier acrylic adhesive.

The combination of Subsurface, Tie bond, and Newly Obtained Active surface were evaluated against known contaminates and recorded. The evaluations reconfirmed various medical research centers and laboratory results.

The material applied to various substrates were evaluated for durability, such as scratch resistance, climate exposure, safety in application, ease of usage and practicality in cleaning and maintenance. Various tie bonds in more demanding applications, with the criteria that the newly obtained surface performs to its intended purpose effectively as a continuous anti-bacterial and anti-viral surface.

The active surfaces were evaluated against known contaminates and recorded.

Combinations of several antibacterial and antiviral surfaces were designed to work in synergy and have a broad range of performances.

The system allows the exposed surface to be made in 3d or designed patterns. In addition, it can be a combination of several antibacterial and antiviral products and methods, such as bioelectric, photocatalytic, moisture absorbing, graphene, in addition to passive/active bacteria, virus neutralizing components, thus creating odorless surfaces to meet wide ranging needs.

According to FIGS. 2 to 5, the invention can be used on any surface like common and/or frequently touched surfaces, such as handles on doors, bed rails, wall bumpers, handrails, shopping carts, food trolleys, sliding doors, moving doors, and various surfaces in different industries such as for example: hotels, restaurants, hospitals, airports, shops, food and grocery delivery, shopping malls, public transport, and other public or private space.

1. An antibacterial and/or antiviral multilayers material comprising:
a tie binding layer (2; 72) with at least one adhesive face (4; 76) arranged to be fixed on any suitable surface (1; 71).
an active treatment layer (3; 73) comprising anti-bacterial and/or anti-viral agents.

2. An antibacterial and/or antiviral multilayers material according to claim 1, wherein the active treatment layer is arranged on the face of the tie binding layer opposite to adhesive face.

3. An antibacterial and/or antiviral multilayers material 70 according to claim 1, wherein the active treatment layer 73 is arranged to cover at least in part the adhesive face 76 of the tie binding layer 72.

4. An antibacterial and/or antiviral multilayers material according to one of claims 1 to 3, wherein the material is in the form of a roll.

5. An antibacterial and/or antiviral multilayers material according to one of claims 1 to 4, wherein a protective film is present on the adhesive face (4) of the tie binding layer.

6. An antibacterial and/or antiviral multilayers material according to one of claims 1 to 5, wherein the active treatment layer comprises at least a metallic agent.

7. An antibacterial and/or antiviral multilayers material according to claim 6, wherein the metallic agent is copper, a copper alloys, silver, brass, bronze, zinc, zinc oxide, titanium dioxide and/or a combination thereof.

8. An antibacterial and/or antiviral multilayers material according to one of claims 1 to 7, wherein the tie binding layer comprises a pressure sensitive adhesive.

9. An antibacterial and/or antiviral multilayers material according to claim 8, wherein the pressure sensitive adhesive comprises PET, PVC, foamed pure acrylic, foamed modified acrylic, solid pure acrylic, polyurethane, polyurea, epoxy, cyanoacrylate, PVA, contact adhesive, hot melt materials, fabric with a rubber adhesive system backing or a combination thereof.

10. Method to apply the antibacterial and/or antiviral multilayers material of any of claims 1 to 9, comprising the steps of:
Placing the multilayers material over the surface to be treated,
firmly and neatly pressing onto the substrate to ensure adhesion of the material to the substrate.

11. Method according to claim 10, further comprising, before spreading the multilayers material over the surface to be treated:
Cleaning the surface to be treated.

12. Use of the antibacterial and/or antiviral multilayers material of claims 1 to 9, to cover a trolley handle, a door handle, an elevator handle, or elevator buttons.

13. Use of the antibacterial and/or antiviral multilayers material of claims 1 to 9, to cover a mop or broom stick.

14. Use of the antibacterial and/ or antiviral multilayers material of claims 1 to 9 as an antiseptic, disinfecting dressing for wounds, where the active materials mentioned are directly or indirectly made to be in contact with wounds.

15. Use of the antibacterial and/or antiviral multilayers material of claims 1 to 9 to produce a biologically clean surface that continuously perpetually acts as a disinfectant.
